(19) United States
(12) Patent Application Publication
(10) Pub. No.: US 2007/0099306 A1
(43) Pub. Date: May 3, 2007

(54) LIQUID CRYSTAL SENSOR SYSTEM

(75) Inventor: Christopher J. WOOLVERTON, Kent, OH (US)

Correspondence Address: HAHN LOESER & PARKS, LLP, One GOJO Plaza, Suite 300, AKRON, OH, 44311-1076, US

(73) Assignee: KENT STATE UNIVERSITY, Kent, OH (US)

(21) Appl. No.: 11/535,541

(22) Filed: Sep. 27, 2006

Related U.S. Application Data
(60) Provisional application No. 60/596,485, filed on Sep. 28, 2005.

(51) Int. Cl. G01N 33/543

(52) U.S. Cl. 436/518; 204/450

(57) ABSTRACT

A method of detecting a ligand comprises providing a receptor for a predetermined ligand. The receptor comprises a conductive material. The receptor is contacted with a sample in the presence of a non-conductive medium. When the ligand is present in the sample, the receptor and the ligand form a receptor-ligand complex that is capable of forming a conductive aggregate. A second receptor is then provided. The second receptor is a receptor for the conductive aggregate, and the second receptor is bound to a pair of electrodes. The electrodes are electrically separated from each other in the absence of the conductive aggregate, and are electrically communicating in the presence of conductive aggregates. The method also includes providing a power source in electrical communication with the electrodes, such that an electric circuit is selectively completed in the presence of conductive aggregates, signaling the detection and capture of the ligand. A cassette for performing this method is also provided.

### CROSS-REFERENCE TO RELATED APPLICATIONS/INCORPORATION BY REFERENCE

The present application claims priority from U.S. provisional application Ser. No. 60/596,485, filed Sep. 28, 2005. The disclosure of application Ser. No. 60/596,485 is hereby incorporated by reference.

### TECHNICAL FIELD

This invention relates to the detection of a ligand by a receptor. More particularly, this invention relates to the detection of biologically relevant ligands, such as pathogenic microbes, using a fluid that is biocompatible and non-denaturing. The use of the biocompatible and non-denaturing fluid permits the flow of ligands, receptors and receptor-ligand complexes. Even more particularly, the invention relates to the use of a liquid crystalline material to create an electrical circuit-based biosensor.

### BACKGROUND OF THE INVENTION

The detection of a ligand by a receptor (for example, detection of a pathogenic agent such as a microbe or toxin by an antibody; or detection of an antibody in blood by another antibody; or binding of a chemical toxin, such as nerve gas, to its receptor) is important in the diagnosis and treatment of individuals exposed to disease-causing or toxic agents. Early detection of pathogenic agents can be a great benefit in either disease prophylaxis or therapy before symptoms appear or worsen.

Every species, strain or toxin of a microbe contains unique surface ligands. Using molecular engineering and/or immunological techniques, receptor molecules, such as antibodies, can be isolated that will bind to these ligands with high specificity. Methods have also been developed where receptors, such as antibodies, are linked to a signaling mechanism that is activated upon binding.

Many available diagnostic tests are antibody based, and can be used to detect either a disease-causing agent or a biologic product produced by the patient in response to the agent. There are currently three prevailing methods of antibody production for recognition of ligands (antigens): polyclonal antibody production in whole animals with recognition for multiple epitopes, monoclonal antibody production in transformed cell lines with recognition for a single epitope (after screening), and molecularly engineered phage displayed antibody production in bacteria with recognition of a single epitope (after screening). Each of these receptor systems is capable of binding and identifying a ligand, but the sensitivity of each is limited by the particular immunoassay detection system to which it is interfaced.

Immunoassays, such as enzyme-linked immunosorbent assay (ELISA), enzyme immunoassay (EIA), and radioimmunoassay (RIA), are well known for the detection of antigens. The basic principle in many of these assays is that an enzyme-, chromogen-, fluorogen-, or radionucleotide-conjugated antibody permits antigen detection upon antibody binding. In order for this interaction to be detected as a color, fluorescence or radioactivity change, significant numbers of antibodies must be bound to a correspondingly large number of antigen epitopes. Use of metal-labeled antibodies is well known in histological applications, such as for tracking ligands in electron microscopy studies.

A system for detecting ligands which utilizes an amplification mechanism such as an antibody embedded liquid crystalline material is provided by U.S. Pat. No. 6,171,802, the disclosure of which is incorporated herein by reference. Liquid crystalline materials that are non-conductive are also known. Liquid crystalline toxicity data are not usually published for generic groups, but are occasionally published for specific products. It has not been previously known, however, to use a non-conductive liquid crystal to form a ligand detection complex of metal-coated receptors (antibodies) bound to their specific ligands, forming a metal coated (electrically conductive) receptor-ligand complex that is capable of forming electrically conductive aggregates.

### SUMMARY OF THE INVENTION

It is therefore, an aspect of the present invention to provide a method for using a non-conductive liquid crystal to facilitate conductive-coated receptors (antibodies) in binding to their specific ligands forming an electrically conductive, coated receptor-ligand complex.

In general, the present invention provides a method of detecting a ligand. The method includes providing a receptor for a predetermined ligand. The receptor includes a conductive material. The receptor is contacted with a sample to be tested, in the presence of a non-conductive medium. When the ligand is present in the sample, the receptor and the ligand form a receptor-ligand complex that is capable of forming a conductive aggregate when the receptor binds to at least one other ligand. A second receptor is then provided. The second receptor is a receptor for the conductive aggregate, and the second receptor is bound to a pair of electrodes. The electrodes are electrically separated from each other in the absence of the conductive aggregate, and are electrically communicating in the presence of conductive aggregates. The method also includes providing a power source in electrical communication with the electrodes, such that an electrical circuit is selectively completed in the presence of conductive aggregates, signaling the detection and capture of the ligand.

The present invention also provides a method for detecting a ligand that includes providing a first receptor, wherein the first receptor is a receptor for at least one predetermined ligand, and wherein the first receptor is coated with an electrically conductive material, contacting the receptor with a sample to be tested, in the presence of a non-conductive medium, wherein the receptor and the at least one predetermined ligand, when present in the sample, form a receptor-ligand complex, and wherein the receptor-ligand complex and at least one other ligand form a conductive aggregate, providing a second receptor, wherein the second receptor is a receptor for the conductive aggregate, and wherein the second receptor is bound to a pair of electrodes, and providing a power source in electrical communication with the electrodes, such that an electrical circuit is selectively completed in the presence of conductive aggregates, signaling the detection and capture of the ligand.

The present invention also provides functional cassette for the detection of ligands. The cassette includes at least one area for receiving a sample to be tested for the presence of one or more ligands, wherein a first receptor is present in the sample receiving area in a non-conductive medium, and wherein each first receptor is a receptor for a predetermined ligand, and further wherein each first receptor is coated with a conductive material and is capable of forming a receptor-ligand complex, and the receptor-ligand complex and at least one other ligand are capable of forming at least one conductive aggregate, one or more transport areas in separate fluid communication with each of the sample receiving areas and a corresponding detection area, wherein the conductive aggregates are capable of being transported to the corresponding detection area, wherein each detection area comprises a pair of electrodes and a second receptor bound to the pair of electrodes, and wherein the second receptor is a receptor for the conductive aggregates, and wherein the electrodes may be placed in electrical communication with a power source such that completion of an electric circuit signals the detection and capture of the predetermined ligand.

In another embodiment of the present invention, a functional cassette having a plurality of channels for the detection of ligands is provided. Each channel of the cassette includes a first front portion, wherein the front portion includes a sample application region for a sample to be tested for the presence of one or more ligands, wherein a first receptor is present in the first front portion in a non-conductive medium, and wherein each first receptor is a receptor for a predetermined ligand, and further wherein each first receptor is coated with a conductive material and is capable of forming a receptor-ligand complex, and the receptor-ligand complex and at least one other ligand are capable of forming at least one conductive aggregates, a second middle portion, wherein an area from the first front portion to the second middle portion define a transport area that includes a primary detection area for receptor-ligand complexes and conductive aggregates, wherein the primary detection area includes a pair of electrodes and a second receptor bound to the pair of electrodes, and wherein the second receptor is a receptor for the conductive aggregates, and wherein the electrodes are placed in electrical communication with a power source such that completion of an electric circuit produces an electrical signal that indicates the detection and capture of the predetermined ligand, and a third end portion, wherein the third end portion includes an area for signal amplification of the electrical signal.

### BRIEF DESCRIPTION OF THE DRAWINGS

FIG. 1 is a top view of a multiplex sensor system according to the present invention which permits the detection of multiple ligands simultaneously;

FIG. 2 is schematic representation of the interaction of ligands and receptors to form conductive aggregates according to the present invention; and

FIG. 3 is a schematic representation of a conductive receptor-ligand aggregate completing an electrical circuit and signaling ligand detection and capture.

### DETAILED DESCRIPTION OF THE INVENTION

As mentioned above, the present invention provides a method of detecting a ligand with a receptor for that ligand. The receptor includes a conductive material, typically a metal, such as gold, iron and the like, coated on or otherwise bound to the surface. Typically, the receptor will be an antibody raised against the ligand. The receptor is contacted with a sample to be tested in a non-conductive medium, such as a liquid crystal material. The medium is preferably also non-toxic, i.e., non-denaturing, to biological materials such as antibodies and ligands. Such a medium may also be referred to as “biocompatible.” Use of a liquid crystalline material as the medium for the receptor, ligand, receptor-ligand complex and aggregate mixture should facilitate receptor-ligand interaction as the ligand and receptor are mixed together.

Any receptor, such as antibodies or biologically engineered receptors for ligands, can be incorporated into the device as long as binding of the ligand to the receptor causes a detectable ligand aggregation and/or distortion (change in conformation) of the receptor. For example, any type of monospecific antibody (polyclonal, monoclonal, or phage displayed) can effectively function as a receptor and, thus, each of those antibody types will be described in the following paragraphs. Although phage-displayed antibodies can be expeditiously modified for identification of new ligands and are used as receptor examples in this patent application, any physically-distortable receptor-ligand interaction is appropriate for the detection component.

Antibody-based antigen detection has been exploited for several decades. Injection of a purified ligand (antigen) into a host animal stimulates the immune system to produce an array of antibodies against various reactive sites on the antigen. Since several lymphocytes are responding to different antigenic epitopes, a multi-specific antibody cocktail (polyclonal) is created and can be purified for antigen detection.

Antibody-producing spleen cells (B lymphocytes) are fused with immortalized myeloma cells to create hybridomas which provide nearly infinite quantities of antibody with a single, defined specificity. Interstrain and even interspecies hybrids of these ‘monoclonal’ antibodies can be generated through genetic engineering techniques. These highly specific antibodies have significant therapeutic potential, as evidenced by the U.S. Food and Drug Administration's approval of the use of mouse-human chimeric antibodies for treatment of selected diseases.

Phage-displayed techniques will be used to isolate single chain chimeric antibodies to various pathogenic agents. The genomic DNA of the B lymphocyte contains the code to produce an antibody to virtually all possible ligands (antigens). In a phage displayed antibody system (PDA), DNA encoding a single chain chimera of the native antibody:hypervariable ligand-binding region is synthesized by joining DNA encoding an antibody heavy chain and DNA encoding an antibody light chain and inserting therebetween DNA encoding a linker region. The desired amino acid sequence of the linker region depends on the characteristics required for any given amplification mechanism. The linker region may have to be able to interact and/or bond to a protein or other substance. Therefore, the polypeptide sequence may have to have, for example, a particular conformation, specifically placed functional groups to induce ionic or hydrogen bonds, or a hydrophobicity that is compatible with the amplification mechanism. Regardless of the type of amplification mechanism, however, the linker region plays a critical role in interfacing the amplification mechanism to the receptor.

An amplification mechanism including liquid crystalline material is utilized to amplify a receptor-ligand complex, thereby detecting the presence of ligands in a sample. A liquid crystal is a state of matter in which molecules exhibit some orientational order but little positional order. This intermediate ordering places liquid crystals between solids (which possess both positional and orientational order) and isotropic fluids (which exhibit no long-range order). Solid crystal or isotropic fluid can be caused to transition into a liquid crystal by changing temperature (creating a thermotropic liquid crystal) or by using an appropriate diluting solvent to change the concentration of solid crystal (creating a lyotropic liquid crystal). Both thermotropic and lyotropic liquid crystals can be used as the amplification mechanism of the device of the present invention. In one embodiment, a chromonic lyotropic liquid crystalline material is used as the amplification component of the device of the present invention.

Among these non-surfactant lyotropic liquid crystals are so-called lyotropic chromonic liquid crystals (LCLCs). The LCLC family embraces a range of dyes, drugs, nucleic acids, antibiotics, carcinogens, and anti-cancer agents. The LCLCs are fundamentally different from the better known surfactant-based lyotropic systems. Without limitation, one difference is that LCLC molecules are disc-like or plank- like rather than rod-like. The polar hydrophilic parts form the periphery, while the central core is relatively hydrophobic. This distinction creates a range of different ordered structures. Individual disc-like molecules may form cylindrical aggregates in water. The LCLCs are assumed to be formed by elongated aggregates, lamellar structures, and possibly by aggregates of other shapes.

Most lyotropic liquid crystals are formed using water as a solvent for biphilic molecules which possess polar (hydrophilic) parts and apolar (hydrophobic) parts. When water is added to biphilic molecules, a bilayer forms as the hydrophobic regions coalesce to minimize interaction with water while enhancing the polar component's interaction with water. The concentration and geometry of the specific molecules define the supramolecular order of the liquid crystal. The molecules can aggregate into lamellae as well as disk-like or rod-like micelles, or, generally, aggregates of anisometric shape. These anisometric aggregates form a nematic, smectic, columnar phase, of either non-chiral or chiral (cholesteric phase) nature. For example, the molecules form a stack of lamellae of alternating layers of water and biphilic molecules, thus giving rise to a lamellar smectic phase.

Lyotropic liquid crystals are usually visualized as ordered phases formed by rod-like molecules in water. A fundamental feature of the surfactant molecules is that the polar hydrophilic head group has an attached flexible hydrophobic tail. There is, however, a variety of other lyotropic systems that are not of the surfactant type, but which can also be successfully used in the present invention.

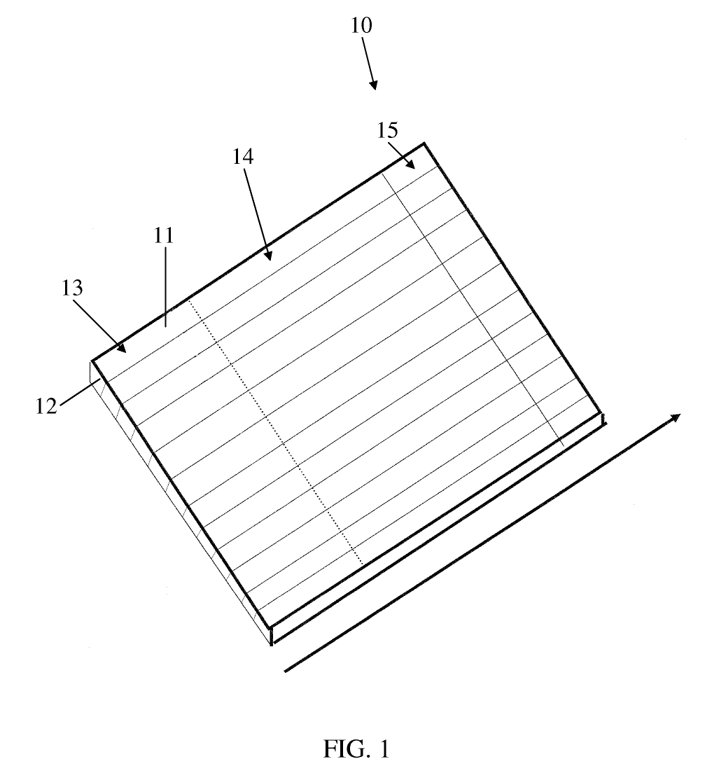

An example of the device of the present invention may be described with reference to the FIGS. 1-3. The device may take form of a cassette 10 having one or more channels 11 between a pair of opposed substrates. As shown in the FIG. 1, the device is a multi-well cassette 10 having one or more channels 11. On a first end of the cassette, is a sample application region 12 for each channel 11. Each channel 11 of cassette 10 has a first front portion 13 that provides for the introduction of at least one receptor, a non-denaturing, non-conductive liquid crystalline material and a sample containing a ligand; a second middle portion 14, wherein area from the first front portion 13 to the second middle portion 14 define a transport area that includes a primary detection area for receptor-ligand complex formation that are capable of being electrically conductive; and a third end portion 15 that provides an area for signal amplification including a transistor. The first detection area and the signal amplification area are in fluid communication.

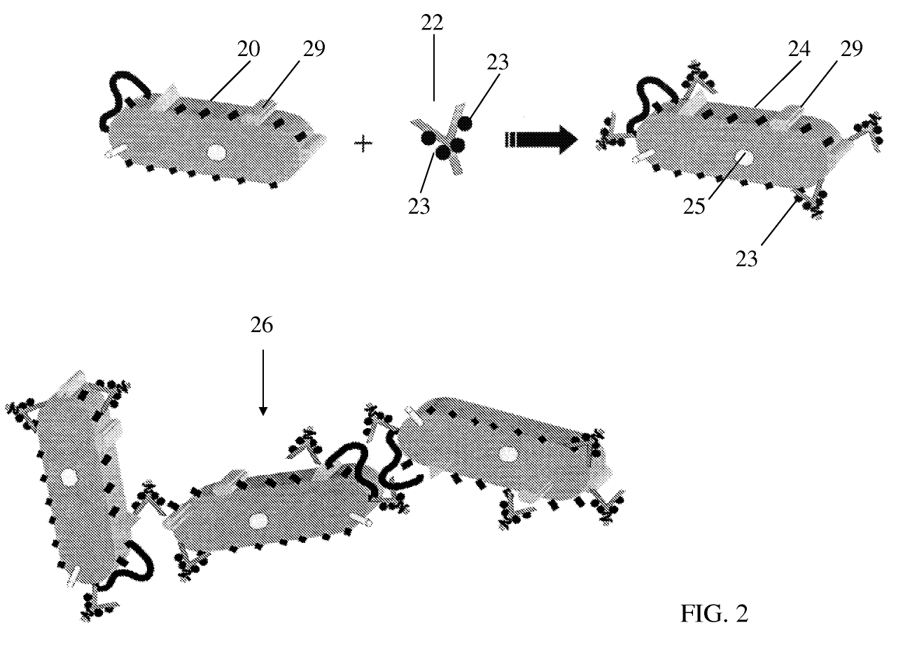

In one embodiment of the present invention, as seen schematically in FIG. 2, when a sample is introduced into the sample application region 12 of a channel 11 of the cassette, a ligand 20 and a ligand-specific receptor 22 can bind together to form a receptor-ligand complex 24. The ligand-specific receptor 22 is capable of binding to multiple ligands 20, therein forming aggregates of receptor-ligand complexes 24.

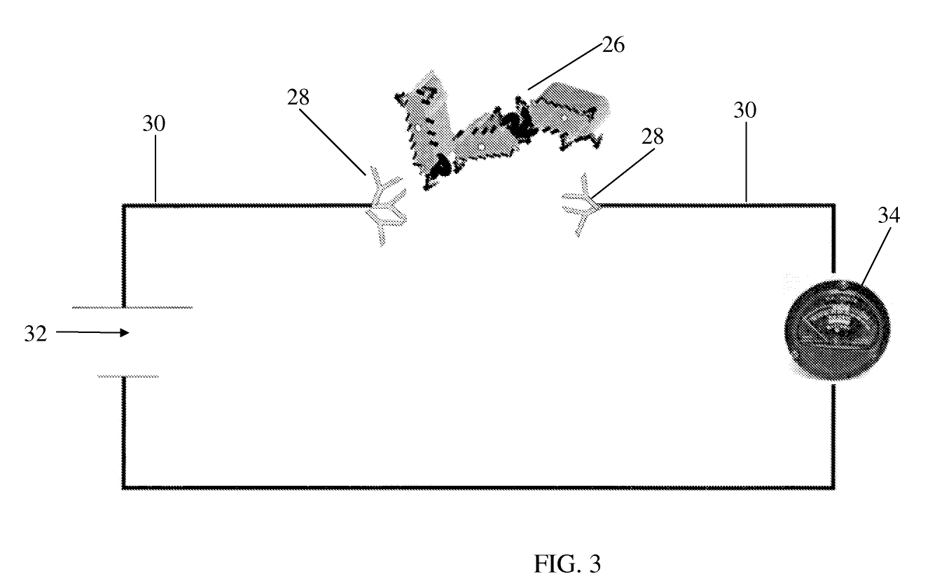

Since the ligand-specific receptor 22 is coated with a conductive material 23, the aggregates of receptor-ligand complexes 26 are electrically conductive. The ligand 20, the ligand-specific receptor 22, the receptor-ligand complexes 24 and the aggregates 26 are bathed in a non-conductive medium, preferably a non-toxic, biocompatible medium such as a lyotropic or thermotropic liquid crystal material. These materials will not conduct electricity. Therefore, it will not be possible to complete an electric circuit absent a separate means for completing that electric circuit. It is for this reason that a second receptor 28, as shown in FIG. 3, which is a receptor for the conductive aggregate 26, provides such means. The binding of the ligand 20 to the first receptor 22 may, for example, bind another ligand 25 with the receptor 21, the conductive aggregate 26, or reveal cryptic binding sites 29 that are recognized by the second receptor 28.

In another embodiment of the present invention, after formation of the aggregates 26, they flow to the second middle portion 14 of the cassette 10, where a second receptor 28 recognizes another ligand or the cryptic receptor sites 23 revealed by the binding of the first receptor 22 to the ligand 20. The second receptor 28 is bound to a pair of electrodes 30. The electrodes are electrically separated from each other in the absence of the conductive aggregate 26, but are electrically communicating in the presence of conductive aggregates 26 because the electrically conductive aggregates 26 bridge the electrodes 30 to complete the circuit. A power source 32 may be placed in electrical communication with the electrodes 30 which are in contact with a metering device 34 for measuring the current. An electric circuit is selectively completed in the presence of conductive aggregates 26, signaling the detection and capture of the ligand. It is envisioned that this invention would be extremely sensitive in that capture of a very small number of ligands could form an aggregate 26 of sufficient size to complete the circuit. As the flow of current proceeds through the circuit as shown in FIG. 3, the resulting electrical signal may be amplified using traditional transistor-based technology or transistor-based nanotechnology.

Based upon the foregoing disclosure, it should now be apparent that device of the present invention will carry out the objects set forth hereinabove. It is, therefore, to be understood that any variations evident fall within the scope of the claimed invention and thus, the selection of specific component elements can be determined without departing from the spirit of the invention herein disclosed and described.

What is claimed is:

1. A method of detecting a ligand, the method comprising:
providing a first receptor, wherein the first receptor is a receptor for at least one predetermined ligand, and wherein the first receptor is coated with an electrically conductive material;
contacting the receptor with a sample to be tested, in the presence of a non-conductive medium, wherein the receptor and the at least one predetermined ligand, when present in the sample, form a receptor-ligand complex, and wherein the receptor-ligand complex and at least one other ligand form a conductive aggregate;
providing a second receptor, wherein the second receptor is a receptor for the conductive aggregate, and wherein the second receptor is bound to a pair of electrodes; and
providing a power source in electrical communication with the electrodes, such that an electrical circuit is selectively completed in the presence of conductive aggregates, signaling the detection and capture of the ligand.

2. The method of claim 1, wherein the non-conductive medium is non-denaturing of biological materials.

3. The method of claim 2, wherein the medium is a liquid crystalline material.

4. The method of claim 3, wherein the liquid crystalline material is a lyotropic liquid crystalline material.

5. The method of claim 3, wherein the liquid crystalline material is a thermotropic liquid crystalline material.

6. The method of claim 1, wherein the first and second receptors are antibodies.

7. The method of claim 1, wherein the pair of electrodes are electrically separated from each other in the absence of the conductive aggregate, and are electrically communicating in the presence of conductive aggregates.

8. A functional cassette for the detection of ligands comprising:
at least one area for receiving a sample to be tested for the presence of one or more ligands, wherein a first receptor is present in the sample receiving area in a non-conductive medium, and wherein each first receptor is a receptor for a predetermined ligand, and further wherein each first receptor is coated with a conductive material and is capable of forming a receptor-ligand complex, and the receptor-ligand complex and at least one other ligand are capable of forming at least one conductive aggregates;
one or more transport areas in separate fluid communication with each of the sample receiving areas and a corresponding detection area, wherein the conductive aggregates are capable of being transported to the corresponding detection area,
wherein each detection area comprises a pair of electrodes and a second receptor bound to the pair of electrodes, and wherein the second receptor is a receptor for the conductive aggregates; and
wherein the electrodes may be placed in electrical communication with a power source such that completion of an electric circuit signals the detection and capture of the predetermined ligand.

9. The cassette of claim 8, wherein the pair of electrodes are electrically separated from each other in the absence of the conductive aggregate and are electrically communicating in the presence of conductive aggregate.

10. The method of claim 8, wherein the non-conductive medium is non-denaturing of biological materials.

11. The method of claim 10, wherein the medium is a liquid crystalline material.

12. The method of claim 11, wherein the liquid crystalline material is a lyotropic liquid crystalline material.

13. The method of claim 11, wherein the liquid crystalline material is a thermotropic liquid crystalline material.

14. The method of claim 8, wherein the first and second receptors are antibodies.

15. A functional cassette having a plurality of channels for the detection of ligands, each channel of the cassette comprising:
a first front portion, wherein the front portion includes a sample application region for a sample to be tested for the presence of one or more ligands, wherein a first receptor is present in the first front portion in a non-conductive medium, and wherein each first receptor is a receptor for a predetermined ligand, and further wherein each first receptor is coated with a conductive material and is capable of forming a receptor-ligand complex, and the receptor-ligand complex and at least one other ligand are capable of forming at least one conductive aggregates;
a second middle portion, wherein an area from the first front portion to the second middle portion define a transport area that includes a primary detection area for receptor-ligand complexes and conductive aggregates, wherein the primary detection area includes a pair of electrodes and a second receptor bound to the pair of electrodes, and wherein the second receptor is a receptor for the conductive aggregates, and wherein the electrodes are placed in electrical communication with a power source such that completion of an electric circuit produces an electrical signal that indicates the detection and capture of the predetermined ligand; and
a third end portion, wherein the third end portion includes an area for signal amplification of the electrical signal.

16. The cassette of claim 15, wherein the first front portion, second middle portion and third end portion are in fluid communication with each other.

17. The cassette of claim 15, wherein the pair of electrodes are electrically separated from each other in the absence of the conductive aggregate and are electrically communicating in the presence of conductive aggregate.

18. The cassette of claim 15, wherein the electrical signal is amplified by a transistor.

19. The method of claim 15, wherein the non-conductive medium is non-denaturing of biological materials.

20. The method of claim 19, wherein the medium is a liquid crystalline material.
